(19) United States
(12) Patent Application Publication
(10) Pub. No.: US 2004/0082856 A1
(43) Pub. Date: Apr. 29, 2004

(54) Support bra for ultrasonic breast scanner

(75) Inventor: Vasilis Z. Marmarelis, Irvine, CA (US)

Correspondence Address: MCDERMOTT, WILL & EMERY, Suite 3400, 2049 Century Park East, Los Angeles, CA, 90067, US

(73) Assignee: Alfred E. Mann Institute for Biomedical Engineering, University of Southern California

(21) Appl. No.: 10/620,739

(22) Filed: Jul. 16, 2003

Related U.S. Application Data
(60) Provisional application No. 60/396,516, filed on Jul. 16, 2002.

(51) Int. Cl. A61B 8/00

(52) U.S. Cl. 600/437

(57) ABSTRACT

A receptacle for supporting a breast during ultrasonic scanning. The receptacle may include a contoured cup made of material that is substantially transparent to acoustical energy and have an open end into which the breast may be inserted and a narrowed end to receive a nipple of the breast. The receptacle may include spaced-apart elongated members, each made of material that is not substantially transparent to acoustical energy and each being mechanically coupled to the open end and to the narrow end of the cup. A fluid pump, impedance matching and a contoured tabletop are also disclosed.

### CROSS-REFERENCE TO RELATED APPLICATION

This application is based upon and claims priority to U.S. Provisional Application Serial No. 60/396,516, entitled “Support Bra for Ultrasonic Breast Scanner,” filed Jul. 16, 2002, the entire content of which is incorporated herein by reference.

### BACKGROUND OF INVENTION

1. Field of Invention

This application relates to breast mammography and, more particularly, ultrasonic breast scanners.

2. Description of Related Art

Breast mammography has traditionally used x-ray techniques. Unfortunately, x-rays can be harmful to the patient. The breast is also frequently compressed during the procedure. This can create discomfort.

Ultrasonic scanning has been proposed as an alternate approach. The breast is dangled in a fluid bath. An ultrasonic transmitter and cooperating ultrasonic receiver are rotated around the breast to scan a series of stacked coronal imaging planes. These stacked planes are then used to generate data representative of a three-dimensional image of the breast tissue.

Unfortunately, the breast may move during the scanning process. This can be caused by turbulence in the fluid and/or by movement of the patient. Such movement can cause blurring of the three-dimensional image. The breast may also secrete fluid creating sanitary issues.

### SUMMARY OF INVENTION

A receptacle for supporting a breast during ultrasonic scanning may include a contoured cup made of material that is substantially transparent to acoustical energy. The cup may have an open end into which the breast may be inserted and a narrowed end configured to receive the nipple of the breast. The receptacle may also have spaced-apart elongated members, each made of material that is not substantially transparent to acoustical energy and each being mechanically coupled to the open end and to the narrowed end of the cup.

At least some of the elongated members may be substantially straight. Each substantially straight member may be mechanically coupled to the cup at a point between the open end and the narrowed end. The point may be approximately midway between the open end and the narrowed end.

Each straight member may be mechanically coupled to the narrowed end by a substantially rigid spacer.

The scanning may create a set of substantially parallel coronal planes, and each substantially-straight member may be substantially perpendicular to these coronal planes.

The cup may be substantially symmetrical about an axis and each substantially straight member may be substantially parallel to that axis.

At least some of the elongated members may be contoured. The contour of each contoured member may be substantially the same as the contour of the cup. Each contoured member may be matingly affixed to the surface of the cup.

At least some of the elongated members may be substantially straight while other elongated members may be contoured. The cup may be substantially symmetrical about an axis. Each substantially straight member may intersect an end point of a line segment that is perpendicular to and passes through the axis. A contoured member may intersect the other end point of the line segment.

The number of substantially straight members may equal the number of contoured members. The substantially straight members and the contoured members may be arranged in an alternating sequence.

The spacing between each neighboring pair of elongated members may be substantially equal.

A receptacle for supporting a breast during ultrasonic scanning may include a contoured cup configured to snuggly fit over the breast without stretching significantly. The cup may be made of a material that does not leak fluid and is substantially transparent to acoustical energy.

The contoured cup may include an elastic polymer. The elastic polymer may include latex.

An acoustically conductive material may be placed on the inside of the cup. The acoustically conductive material may include a viscous gel.

A receptacle for insertion through an opening in an ultrasonic scanner and for supporting a breast during scanning may include a contoured cup made of material that is substantially transparent to acoustical energy that has an open end into which the breast may be inserted and a narrowed end configured to receive the nipple of the breast. The receptacle may also include an annular ring mechanically coupled to the open end of the cup and configured to releasably engage the opening in the ultrasonic scanner.

The annular ring may have a surface that is substantially perpendicular to the contour of the cup at the open end.

The diameter of the open end of the cup may be slightly less than the diameter of the opening in the ultrasonic scanner and the outer diameter of the annular ring may be greater than the diameter of the opening in the ultrasonic scanner.

The annular ring may be flat.

An ultrasonic scanner for scanning a breast may include a rotatable mechanism configured to rotate around the breast. It may also include at least one ultrasonic transducer mechanically coupled to the rotatable mechanism. It may also include a pump configured to cause fluid to flow across the surface of the breast, or across a contoured cup in which the breast is inserted, from approximately the portion of the breast that is closest to the chest to approximately the nipple of the breast, as the rotatable mechanism rotates.

The pump may include a rotatable chamber and a substantially helical groove on the inner wall of the rotatable chamber.

An ultrasonic scanner for scanning a breast may include a rotatable chamber configured to rotate around the breast. The scanner may include at least one ultrasonic transducer mechanically coupled to the rotatable mechanism that has an acoustic impedance. The scanner may include fluid within the rotatable chamber, a contoured cup configured to contain the breast, and gel on the inside of the cup, all having an acoustic impedance substantially the same as the ultrasonic transducer.

These as well as still further features, objects and benefits will now become clear upon an examination of the following Detailed Description of Illustrative Embodiments and the attached drawings.

### BRIEF DESCRIPTION OF DRAWINGS

FIG. 1 shows portions of an ultrasonic scanner with a support bra and contoured coronal plane locator wires.

FIG. 2 shows a support bra with straight and contoured coronal plane locator wires.

FIG. 3 is a top view of FIG. 2.

FIG. 4 is a sectional view of a rotatable chamber using a helical groove as a fluid pump.

FIG. 5 illustrates a contoured tabletop that may be used in an ultrasonic scanner.

### DESCRIPTION OF ILLUSTRATIVE EMBODIMENTS

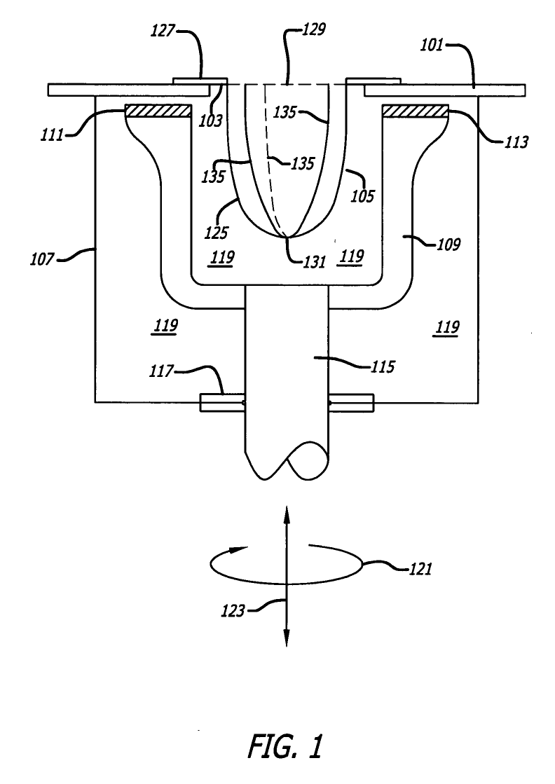

FIG. 1 shows portions of an ultrasonic scanner with a support bra and contoured coronal plane locator wires.

As shown in FIG. 1, a tabletop 101 on which a female patient may lie may include an opening 103 into which a bra-like receptacle 105 may be inserted. Directly below the opening 103 may be a stationary chamber 107.

The tabletop 101 may be long enough and wide enough to accommodate varies sizes of female patients. Similarly, the opening 103 may be large enough to accommodate various sizes of breasts that will be placed within it. A reduction ring (not shown) may be inserted in the opening 103 to better support female subjects with smaller breasts.

The receptacle 105 may include a contoured cup 125 attached to an annular ring 127.

The contoured cup 125 may include an open end 129 into which the breast may be inserted and a narrowed end 131 into which the nipple of the breast may be inserted.

The contoured cup 125 may be made of material that is transparent to acoustical energy, such as an elastic polymer, such as latex.

The contoured cup may be of a size that snugly fits the breast without having to stretch significantly to accommodate the breast, thus minimizing the degree to which the contoured cup 125 compresses the breast. In practice, this may require different sizes of the contoured cup 125 to be produced and made available, so that different sizes of breasts may be correctly fitted.

The annular ring 127 that is affixed to the contoured cup 125 may be a flat surface that is substantially perpendicular to the upper wall of the contoured cup 125. It may have an inner diameter that is smaller than the diameter of the opening 103 and an outer diameter that is larger than the diameter of the opening 103. Using this configuration, the receptacle 105 can easily be inserted within the opening 103 and allowed to have its annular ring 127 rest on the top of the table 101. After being used, the receptacle 105 can be removed from the opening 103 and, if desired, discarded or cleaned. A new or cleaned receptacle 105 can then be easily used for the next subject.

Before the breast is inserted in the contoured cup 125, the inner wall of the contoured cup 125 may be coated with a gel. The gel may be acoustically conductive. The gel may be spread on all inner surfaces of the cup 125 to insure that there are no air pockets between the breast and the contoured cup 125. The gel may also be viscous to insure that the breast does not move with respect to the contoured cup 125 during the scanning process.

Within the stationary chamber 107 may be a rotatable chamber 109 to which may be affixed an ultrasonic transmitter 111 and an ultrasonic receiver 113.

The ultrasonic transmitter 111 may consist of only a single element or may include an array of elements. Similarly, the ultrasonic receiver 113 may consist of only a single element or may include an array of elements.

Both the stationary chamber 107 and the rotatable chamber 109 may be filled with a fluid 119. The contoured cup 125 may be completely sealed to prevent fluid excreted by the breast from mixing with the fluid 119.

The fluid 119 may be of a type that provides acoustic coupling. It may also be of low viscosity to minimize turbulence during rotation of the rotatable chamber 109. Water is one example of a fluid that may be used.

The materials used for the fluid 119, the contoured cup 125 and the gel may be selected so that their acoustic impedances closely match the acoustic impedances of the ultrasonic transmitter 111 and the ultrasonic receiver 113.

The impedance of normal breast tissue may be significantly different from the acoustic impedances of the ultrasonic transmitter 111 and the ultrasonic receiver 113. In this event, the materials used for the fluid 119, the contoured cup 125 and the gel may be selected so as to have acoustic impedances that bridge the impedance difference between the ultrasonic transducers and the normal breast tissue in successive steps.

Appropriate fluid filling and drainage mechanisms (not shown) may be employed to fill the chambers 107 and 109 with fluid, in preparation for ultrasonic scanning, and to then remove the fluid after scanning.

The filling mechanism (not shown) may deliver sufficient fluid to cause the level of the fluid in the stationary chamber 107 to exceed the level of the ultrasonic transmitter 111 and ultrasonic receiver 113. Holes (not shown) may be placed in the walls of the rotatable chamber 109 to allow fluid to freely flow between the rotatable chamber 109 and the stationary chamber 107. The inner walls of the rotatable chamber 109 may be configured to minimize turbulence during its rotation.

The rotatable chamber 109 may be affixed to a shaft 115 that extends beneath the stationary chamber 107 through a fluid tight bearing seal 117.

The rotatable chamber 109 may be rotated around the receptacle 105, as reflected by a rotational movement arrow 121. It may also be lowered during the scanning process, as reflected by a longitudinal movement arrow 123. These movements may be accomplished by the application of appropriate forces to the shaft 115 by an appropriate drive mechanism (not shown).

The exact motion imparted to the shaft 115 and, in turn, to the rotatable chamber 109 can vary. In one application, the scanning process may begin by the rotatable chamber 109 being raised to its highest position such that the top of the ultrasonic transmitter 111 and the top of the ultrasonic receiver 113 are as high as possible without scraping the underneath side of the tabletop 101. The shaft 115 may then be rotated to cause the rotatable chamber 109 to rotate 360 degrees.

During this rotation, an ultrasonic signal may be directed from the ultrasonic transmitter 111 through the fluid 119, the receptacle 105 and the breast that is within the receptacle, until it received by the ultrasonic receiver 113. Data representing a two dimensional, coronal image plane cross-section of the breast may then be gathered.

After this first coronal plane is scanned, the drive mechanism (not shown) to which the shaft 115 is attached may incrementally lower the shaft, causing a corresponding incremental lowering of the rotatable chamber 109. The drive system may then rotate the shaft 115 through another 360 degrees, causing a second coronal plane to be scanned. The process may then repeat until all of the breast has been scanned.

Shaft 115 may instead be continually lowered while it is being rotated, resulting in a helical scan.

Regardless of which approach is used, the stacked coronal imaging planes may then be analyzed in accordance with well known techniques to generate data that represents a three-dimensional image of the tissue in the breast.

Coronal plane locators 135 may also be used. The locators 135 may each include an elongated member affixed at its upper end to the edge of the opening 129 in the contoured cup 125. They may also be affixed at their lower end to the narrowed portion 131 of the contoured cup 125. They may also be affixed at some or all of the points in between to the contoured cup 125. The locators 135 may also be contoured to match the contour of the contoured cup 125.

The locators 135 may be of material that is substantially opaque to ultrasonic signals. For example, the locators 135 may be thin metal wires.

The locators 135 may be spaced apart. They may be equally spaced around the perimeter wall of the contoured cup 125, such as the 120-degree spacing shown in FIG. 1.

Although three locators 135 are shown in FIG. 1, it is to be understood that a different number could be used instead, such as two or four.

The acoustically opaque nature of the locators 135 will cause them to appear in each coronal imaging plane. In turn, their presence in all of the coronal imaging planes can be used by the processing system (not shown) to aid in the co-registration of these planes. These location points may also aid in correcting any errors caused by motion of the breast during the scanning process. An auto-focusing processing algorithm, such as is used in synthetic aperture radar (SAR), may be used for this purpose.

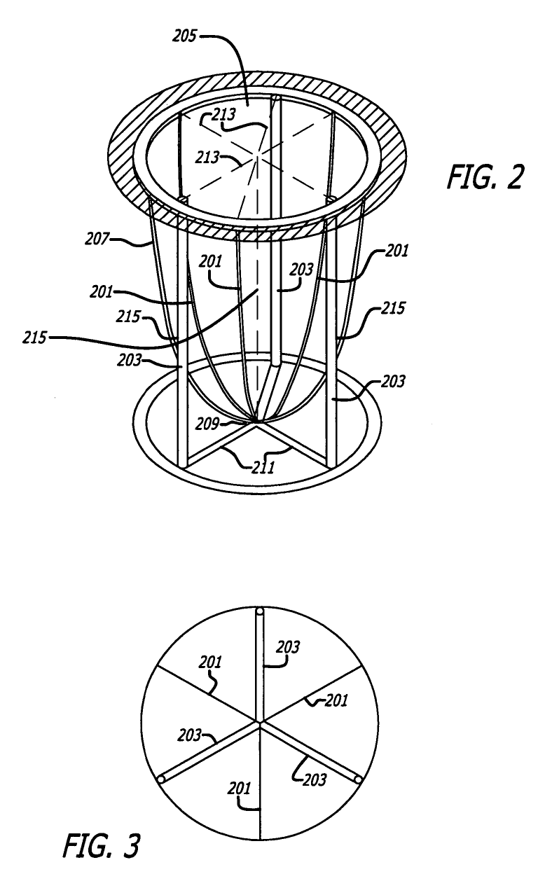

FIG. 2 shows a support bra with straight and contoured coronal plane locator wires. FIG. 3 is a top view of FIG. 2.

As shown in FIGS. 2 and 3, elongated contoured locators 201 may be used. These may be just like the locators 135 that were discussed above in connection with FIG. 1.

Elongated straight locators 203 may also be used. These may similarly be attached at one end to the perimeter of the opening 205 of the contoured cup 207 and, at their other end, to the narrowed end 209 of the contoured cup 207. The attachment of the straight locators 203 at their lower end to the narrowed portion 209 of the contoured cup 207 may be facilitated by a substantially rigid spacer, such as the radial arms 211.

As with the contoured locators 135 shown in FIG. 1, the contoured locators 201 shown in FIGS. 2 and 3 may be evenly spaced around the perimeter of the opening 205. Similarly, straight locators 203 may be evenly spaced around the perimeter of the opening 205.

As perhaps most clearly illustrated in FIG. 3, the locators around the perimeter of the opening 205 may alternate between a contoured locator 201 and a straight locator 203. The upper end of each straight locator 203 may be on a line segment 213 that passes through the symmetric axis 215 of the contoured cup 207, while the upper end of a curved locator 201 may be attached to the other end of that line segment. The straight locators 203 may also be attached at their approximate midpoints to the contoured cup 207.

As with the contoured locators 125 in FIG. 1, there can be any number of contoured locators 201 and straight locators 203 in FIGS. 2 and 3. Although shown as having both straight and contoured locators, it is also to be understood that the receptacle may include only contoured locators, as shown in FIG. 1, only straight locators, or no locators.

As with the contoured locators 135 in FIG. 1, the contoured locators 201 and/or the straight locators 203 in FIGS. 2 and 3 may be used to aid in co-registering the stacked coronal planes that result from the scan.

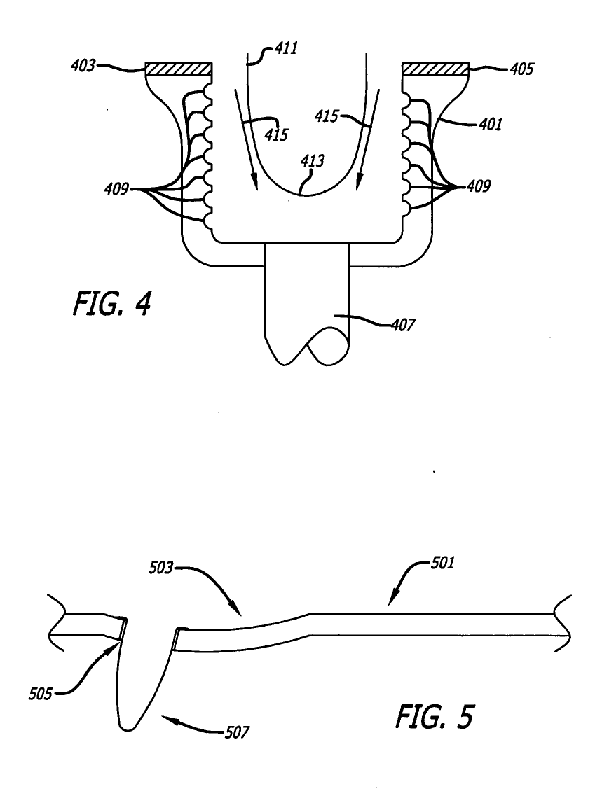

FIG. 4 is a sectional view of a rotatable chamber using a helical groove as a fluid pump. As shown in FIG. 4, a rotatable chamber 401 may have affixed at its top an ultrasonic transmitter 403 and an ultrasonic receiver 405. A shaft 407 may be affixed to the rotatable chamber 401 and may be controlled as described above in connection with the shaft 115 in FIG. 1.

The rotatable chamber 401 may include a helical groove 409 in its inner wall. When the rotatable chamber 401 is filled with fluid and rotates, the rotating chamber 401 may cooperate with the helical groove 409 to act like a pump and can cause fluid within the chamber to swirl in a manner that causes the fluid to flow from the portion of the cup 411 that is closest to the chest of the patient to the portion of the cup 413 that is in the area of the nipple of the breast, as reflected by fluid flow arrows 415. This may help stabilize the breast during the scanning process. The fluid pumping action may also be used in systems in which the breast is inserted without a bra-like receptacle.

Other types of fluid pumps may be used instead, such as a pump that is external to the rotatable chamber 401 that delivers fluid into the rotating chamber and in a manner that similarly swirls downwardly across the breast, as shown by the fluid flow arrows 415.

FIG. 5 illustrates a contoured tabletop that may be used in an ultrasonic scanner. As shown in FIG. 5, a tabletop 501 containing an opening 505 in which a receptacle 507 is inserted. The tabletop 501, opening 505 and receptacle 507 may be governed by the same considerations that were discussed above in connection with the tabletop 101 opening 103 and receptacle 105 shown in FIG. 1.

The tabletop 501 may also include a contoured section 503. The contour of the contoured section 503 may substantially match the contour of the average chest of the patient that lies on top of it. This may increase the comfort to the patients and help insure that the breast of each patient is always inserted through the opening 505 in the same X-Y orientation, thus insuring consistency in the orientation of the tissue images that are developed.

Having now described illustrative embodiments, those skilled in the art will appreciate that modifications may be made to them without departing from the spirit of the concepts that are embodied in them. Further, it is not intended that the scope of this application be limited to these specific embodiments or to their specific features or benefits. Rather, it is intended that the scope of this application be limited solely to the claims which now follow and to their equivalents.

I claim:

1. A receptacle for supporting a breast during ultrasonic scanning comprising:
a contoured cup made of material that is substantially transparent to acoustical energy and having an open end into which the breast may be inserted and a narrowed end configured to receive the nipple of the breast; and
spaced-apart elongated members, each made of material that is not substantially transparent to acoustical energy and each being mechanically coupled to the cup.

2. The receptacle of claim 2 wherein at least some of the elongated members are substantially straight.

3. The receptacle of claim 2 wherein each substantially straight member is mechanically coupled to the open end and to the narrowed end of the cup.

4. The receptacle of claim 3 wherein each substantially straight member is also mechanically coupled to the cup at a point that is approximately midway between the open end and the narrowed end.

5. The receptacle of claim 2 wherein each straight member is mechanically coupled to the narrowed end by a substantially rigid spacer.

6. The receptacle of claim 3 wherein the scanning creates a set of substantially parallel coronal planes and wherein each substantially-straight member is substantially perpendicular to the coronal planes.

7. The receptacle of claim 3 wherein the cup is substantially symmetrical about an axis and wherein each substantially straight member is substantially parallel to the axis.

8. The receptacle of claim 1 wherein at least some of the elongated members are contoured.

9. The receptacle of claim 8 wherein the contour of each contoured member is substantially the same as the contour of the cup.

10. The receptacle of claim 9 wherein each contoured member is matingly affixed to the contour of the cup.

11. The receptacle of claim 1 wherein at least some of the elongated members are substantially straight and wherein at least some of the elongated members are contoured.

12. The receptacle of claim 11 wherein the cup is substantially symmetrical about an axis, wherein each substantially straight member intersects an end point of a line segment that is perpendicular to and passes through the axis, and wherein a contoured member intersects the other end point of the line segment.

13. The receptacle of claim 12 wherein there are an equal number of substantially straight and contoured members.

14. The receptacle of claim 11 wherein the substantially straight members and the contoured members are arranged in an alternating sequence.

15. The receptacle of claim 1 wherein the spacing between each neighboring pair of elongated members is substantially equally.

16. A receptacle for supporting a breast during ultrasonic scanning comprising a contoured cup configured to snuggly fit over the breast without stretching significantly and being made of a material that does not leak fluid and is substantially transparent to acoustical energy.

17. The receptacle of claim 16 wherein the contoured cup includes an elastic polymer.

18. The receptacle of claim 17 wherein the elastic polymer includes latex.

19. The receptacle of claim 16 further including an acoustically conductive material on the inside of the cup.

20. The receptacle of claim 17 wherein the acoustically conductive material includes a viscous gel.

21. A receptacle for insertion through an opening in an ultrasonic scanner and for supporting a breast during scanning comprising:
a contoured cup made of material that is substantially transparent to acoustical energy and having an open end into which the breast may be inserted and a narrowed end configured to receive the nipple of the breast; and
an annular ring mechanically coupled to the open end of the cup and configured to releasably engage the opening in the ultrasonic scanner.

22. The receptacle of claim 21 wherein the annular ring has a surface that is substantially perpendicular to the contour of the cup at the open end.

23. The receptacle of claim 21 wherein the diameter of the open end of the cup is slightly less than the diameter of the opening in the ultrasonic scanner and wherein the outer diameter of the annular ring is greater than the diameter of the opening in the ultrasonic scanner.

24. The receptacle of claim 21 wherein the annular ring is flat.

25. An ultrasonic scanner for scanning a breast comprising:
a rotatable mechanism configured to rotate around the breast;
at least one ultrasonic transducer mechanically coupled to the rotatable mechanism; and
a pump configured to cause fluid to flow across the surface of the breast or the surface of a contoured cup in which the breast is inserted as the rotatable mechanism rotates from approximately the portion of the breast or cup that is closest to the chest to approximately the nipple of the breast or portion of the cup that surrounds it.

26. The ultrasonic scanner of claim 25 wherein the pump includes a rotatable chamber and a substantially helical groove on the inner wall of the rotatable chamber.

27. An ultrasonic scanner for scanning a breast comprising:
a rotatable chamber configured to rotate around the breast;
at least one ultrasonic transducer mechanically coupled to the rotatable mechanism and having an acoustic impedance;
fluid within the rotatable chamber having an acoustic impedance substantially the same as the ultrasonic transducer;
a contoured cup configured to contain the breast and having an acoustic impedance substantially the same as the ultrasonic transducer; and
gel on the inside of the cup having an acoustic impedance substantially the same as the ultrasonic transducer.

28. An ultrasonic scanner having a tabletop, an opening in the tabletop configured to accommodate a breast, and a contoured surface surrounding the opening configured to substantially match the curvature of portions of the chest of a patient.
